(19) United States
(12) Patent Application Publication
(10) Pub. No.: US 2005/0251124 A1
(43) Pub. Date: Nov. 10, 2005

(54) Apparatus and method for cryosurgery within a body cavity

(75) Inventors: Roni Zvuloni, Haifa (IL); Yan Moore, Uniondale, NY (US); Paul Sofer, Zofit (IL); James McGlone, Garden City, NY (US)

Correspondence Address: Martin Moynihan;c/o Anthony Castorina, Suite 207, 2001 Jefferson Davis Highway, Arlington, VA, 22202, US

(73) Assignee: Galil Medical Ltd., Yokneam (IL)

(21) Appl. No.: 11/185,699

(22) Filed: Jul. 21, 2005

Related U.S. Application Data
(62) Division of application No. 10/151,310, filed on May 21, 2002.
(60) Provisional application No. 60/291,990, filed on May 21, 2001.
(60) Provisional application No. 60/300,097, filed on Jun. 25, 2001.

(51) Int. Cl. A61B 18/02

(52) U.S. Cl. 606/21

(57) ABSTRACT

Apparatus and method for cryosurgery within a body cavity are disclosed. The apparatus includes a trocar installable in an external passageway opened in a wall of a body cavity of a patient, the trocar having a portal serving to maintain and control the external passageway after installation of the trocar, the portal being useable for transmitting therethrough at least one surgical instrument for use during a surgical procedure. The apparatus further includes at least one cryoprobe deployable through the portal of the trocar into a body cavity. The cryoprobe is operable to be positioned in the body cavity in a selected orientation and position, and to cryoablate a tissue within the body cavity when in that selected orientation and position.

### RELATED APPLICATIONS

This application is a Divisional of U.S. patent application Ser. No. 10/151,310, filed May 21, 2002, which claims the benefit of priority from U.S. Provisional Patent Application No. 60/291,990, filed May 21, 2001, and U.S. Provisional Patent Application No. 60/300,097, filed Jun. 25, 2001, the disclosure thereof is incorporated herein by reference.

### FIELD AND BACKGROUND OF THE INVENTION

The present invention relates to an apparatus and method for cryosurgery within a body cavity. More particularly, the present invention relates to an apparatus including a trocar having an edge shaped for penetrating an external wall of a body cavity, thereby opening an external passageway into that body cavity, and also having a portal serving to maintain and control that external passageway after installation of the trocar, the portal being useable for transmitting therethrough at least one surgical instrument for use during a surgical procedure. The apparatus further includes at least one cryoprobe deployable through a portal of the trocar into the body cavity, which cryoprobe is operable to be positioned in the body cavity in a selected orientation and position, and is further operable to cryoablate a tissue within said body cavity when in said selected orientation and position.

Cryosurgical procedures involve deep tissue freezing which results in tissue destruction due to rupture of cells and or cell organelles within the tissue. Deep tissue freezing is effected by insertion of a tip of a cryosurgical device into the tissue, and formation of, what is known in the art as, an ice-ball around the tip. Deep freezing of tissues has come to be seen as a minimally invasive surgical technique of choice for a variety of conditions requiring ablation of tissues, having the advantage of minimal destruction of healthy tissues outside the pathological site. In this respect, cryosurgery has been found to be superior to other tissue ablation techniques, for a variety of conditions.

According to methods of prior art, cryosurgery has typically been performed through open surgery, percutaneously (e.g., transperineally), endoscopically or laparoscopically.

In very general terms, two methods have typically been used to effect cryoablation of tissues in the human body.

In a first general method, one or more cryoprobes having a sharp edge are caused to penetrate directly into the body or into an organ of the body, passing through skin, fat, and muscle or other tissue until they have reached a tissue whose cryoablation is desired, at which point the cryoprobes are cooled to effect cryoablation.

In a second general method, endoscopic procedures are used to guide one or more cryoprobes through a naturally occurring body conduit, for example through a urethra or a colon, until a point is reached where cryoablation is required.

U.S. Pat. No. 6,142,991 to Schatzberger is an example of a sophisticated cryosurgery device using an adaptation of the first general method referred to above. Schatzberger's device is designed to affect transperineal cryoablation of the prostate. The cryoprobe or cryoprobes controlled by the device taught by Schatzberger penetrate the body through the perineum, and their (one-dimensional) forward and backward motion can be controlled by a surgeon who manipulates a portion of the cryosurgery device external to the body.

Schatzberger's apparatus comprises (a) a plurality of cryosurgical probes of small diameter, the probes serve for insertion into the patient's organ, the probes being for producing ice-balls for locally freezing selected portions of the organ; (b) a guiding element including a net of apertures for inserting the cryosurgical probes therethrough; and (c) an imaging device for providing a set of images, the images being for providing information on specific planes located at specific depths within the organ, each image including a net of marks correlated to the net of apertures of the guiding element, wherein the marks represent the locations of ice-balls which may be formed by the cryosurgical probes when introduced through the apertures of the guiding element to distinct depths within the organ.

A further example of an advanced cryosurgery device, utilizing the first general method for delivering cryoprobes to a cryoablation site, is provided by U.S. patent application Ser. No. 09/860,486, filed May 21, 2001, by Schatzberger and Zvuloni.

Schatzberger and Zvuloni teach penetration of cryoprobes into the body in two phases. Schatzberger and Zvuloni describe an introducer having a sharp edge capable of penetrating tissues, and an encapsulating sheath containing one or more cryoprobes. In a first phase, the introducer is used to penetrate body tissues until a point is reached which is proximate to a body area where cryoablation is desired. In a second phase, cryoprobes comprising shape memory alloy material are extended from the encapsulating sheath into the surrounding tissue, and there are cooled to effect cryoablation.

An example of the second general method used to effect cryoablation of tissues in the human body, utilizing endoscopic penetration of a naturally occurring body conduit to deliver a cryoprobe to a cryoablation site, is provided by U.S. Pat. No. 6,179,831 to Bliweis. Bliweis teaches a method for treating benign prostate hyperplasia in which a cystoscope is inserted into a prostatic urethra portion of a urethra of a patient having benign prostate hyperplasia, a cryoprobe having an operating tip is guided through a channel of the cystoscope to an portion of the prostatic urethra, the operating tip of the cryoprobe is navigated through a wall of the prostatic urethra into a selected portion of a prostate of the patient, and the cryoprobe is operated to cool operating tip and produce an ice-ball of prostate tissue around the operating tip, locally freezing a portion of the prostate, yet substantially avoiding freezing the prostatic urethra.

The two general cryoablation methods outlined above have proven effective for cryoablation of tissues at various sites within the body. Yet, some pathological tissue sites exist which cannot be adequately treated using those two general methods, either because those methods do not provide access to these sites, or because those methods do not enable adequate monitoring of the placement of cryoprobes with respect to the pathological tissues and do not enable adequate monitoring of the cryoablation procedure during the freezing process. The anterior face of the kidney, for example, cannot adequately be accessed using either of the above-mentioned methods, nor can various other potentially pathological sites within the abdominal and other body cavities. The adrenal glands and the liver portal are additional examples of sites which cannot adequately be accessed using either of the above-mentioned methods. Sites not accessible using these techniques generally include those which are not located close to an accessible body surface, and which are not located within, nor adjacent to, a natural conduit accessible from outside the body, such as a urethra or a colon.

In addition, it is sometimes desirable to utilize several cryoprobes to ablate a single tumor, those several cryoprobes approaching a same tumor from different sides and at different angles. Prior art methods do not adequately provide a means for performing such a multi-probe multi-angle cryoablation, nor do they provide for true freedom of motion of a cryoprobe introduced into the body of a patient.

It is to be noted in particular that whereas Schatzberger, in U.S. Pat. No. 6,142,991 refers to a “flexible connecting line 54” in his FIG. 6a, (substantially reproduced hereinbelow as FIG. 2), the flexibility contemplated is that of a gas supply line external to the body of a patient, which gas supply line transports gas between a source of pressurized gas and a rigidly mounted cryoprobe, having only one degree of freedom of movement once (that of forward and backward motion, only) once inserted into the body of a patient.

Thus there is a widely felt need for, and it would be highly desirable to have, a method and apparatus for performing cryoablation within a body cavity such as the abdominal cavity, which method and apparatus will enable placement of cryoprobes within substantially all regions of the cavity, and in a variety of orientations, and which will allow general freedom of movement of a cryoprobe under control of a surgeon.

There is, further, a widely felt need for, and it would be highly desirable to have, a method and apparatus for performing cryoablation within a body cavity, which enable placement of multiple cryoprobes to effect cryoablation at one or more ablation sites within the cavity, and which will enable use of those cryoprobes in coordination with additional surgical tools, particularly with tools useable to position cryoprobes in a context of body tissues, tools useable to monitor placement of cryoprobes with respect to selected body tissues, and tools useable to monitor the progress of a cryoablation procedure as that procedure takes place.

A popular technique in laparoscopic surgery consists of inflating a body cavity, such as the abdominal cavity, with gas under pressure, thereby expanding the volume of that cavity, creating space for a surgeon to conveniently manipulate surgical tools, and facilitating his use of optical or electronic instruments for making visual observations of a surgical site during a surgical procedure. Cryosurgery devices known to prior art do not provide means for utilization of this inflation technique during cryosurgery in a body cavity.

Thus, there is a widely felt need for, and it would be highly desirable to have, a method and apparatus for performing cryoablation within a body cavity such as the abdominal cavity, which method and apparatus enable inflating the body cavity with a gas under pressure, and maintaining that inflated state during a cryosurgery procedure.

### SUMMARY OF THE INVENTION

According to one aspect of the present invention there is provided a cryosurgery apparatus for cryoablation of a tissue of a body, comprising at least one trocar installable in an external passageway opened in a wall of a body cavity, the trocar comprises a portal serving to maintain and control said external passageway after installation of the trocar, the portal being useable for transmitting therethrough at least one surgical instrument for use during a surgical procedure, and at least one cryoprobe deployable through the portal of the trocar into the body cavity, operable to be positioned in the body cavity in a selected orientation and position, and further operable to cryoablate a tissue within the body cavity when in the selected orientation and position.

According to further features in preferred embodiments of the invention described below, the trocar of the cryosurgery apparatus further comprises an edge shaped for penetrating the external wall of the body cavity, the edge being useable to open the external passageway through the wall into the body cavity, thereby enabling to install the trocar in the opening of the wall.

According to still further features in the described preferred embodiments, the trocar further comprises a removable cutting module having an edge shaped for penetrating the external wall of the body cavity, the edge being useable to open the external passageway through the wall into the body cavity, thereby enabling to install the trocar in the opening of the wall.

According to still further features in the described preferred embodiments, the portal is further operable to impede movement of gasses from within the body cavity through the external passageway, thereby enabling to maintain a pressure differential between gasses within the body cavity and gasses outside the body cavity.

According to still further features in the described preferred embodiments, the trocar further comprises a removable pressure-retaining module operable to impede movement of gasses from within the body cavity through the external passageway, thereby enabling to maintain a pressure differential between gasses within the body cavity and gasses outside the body cavity. The pressure-retaining module may comprise one or more diaphragms.

According to still further features in the described preferred embodiments, the cryoprobe comprises a distal portion which comprises a tissue-cooling surface operable to cool a body tissue adjacent to the tissue-cooling surface and a cooling device for cooling the tissue-cooling surface, and also comprises a substantially flexible medial portion designed and constructed for insertion through the portal into the body cavity.

According to still further features in the described preferred embodiments, the cooling device comprises at least one Joule-Thomson heat exchanger and at least one heat exchanging configuration. The Joule-Thomson heat exchanger may also be operable to heat the tissue-cooling surface.

According to still further features in the described preferred embodiments, the distal portion comprises a first conduit for conducting a compressed gas to the Joule-Thompson heat exchanger for use therein, and further comprises a second conduit for conducting a gas decompressed by use in the Joule-Thomson heat exchanger away from the Joule-Thompson heat exchanger. The distal portion may further comprise a distal heat exchanging configuration for transferring heat between the first conduit and the second conduit. The medial portion may also comprise a heat exchanging configuration for transferring heat between the first conduit and the second conduit. The medial heat exchanging configuration may be designed and constructed to be positioned outside the body of a patient during use.

According to still further features in the described preferred embodiments, the distal portion further comprises a distal heat exchanging configuration for exchanging heat between a first gas contained in the first conduit and a second gas contained in the second conduit. The distal heat exchanging configuration is operable to transfer heat from a more highly compressed gas in the first conduit to a less highly compressed gas in the second conduit, and may also be operable to transfer heat from a less highly compressed gas in the second conduit to a more highly compressed gas in the first conduit.

According to still further features in the described preferred embodiments, the medial portion comprises a first medial conduit for transmitting a gas from at least one source of compressed gas to the distal portion. Preferably, the medial portion comprises a first medial conduit for transmitting a gas from a plurality of sources of compressed gas to the distal portion. The apparatus may further comprise a valve for controlling transmission of gas from the at least one source of compressed gas to the distal portion, which valve may be operated by a computer processor under programmed control.

According to still further features in the described preferred embodiments, the medial portion further comprises a second medial conduit for transmitting a gas from the distal portion to a gas receiving unit positioned outside the body cavity.

According to still further features in the described preferred embodiments, the first medial conduit is operable to supply to the distal portion a cooling gas useable in the Joule-Thomson heat exchanger to cool the tissue-cooling surface.

According to still further features in the described preferred embodiments, the first medial conduit is operable to supply to the distal portion a heating gas useable in the Joule-Thomson heat exchanger to heat the tissue-cooling surface.

Preferably, the first medial conduit is operable to supply to the distal portion a cooling gas useable in the Joule-Thomson heat exchanger to cool the tissue-cooling surface, and is also operable to supply to the distal portion a heating gas useable in the Joule-Thomson heat exchanger to heat the tissue-cooling surface.

According to still further features in the described preferred embodiments, the substantially flexible medial portion of the cryoprobe is sufficiently long and sufficiently flexible to enable the cryoprobe, when inserted through the portal into the body cavity, to be freely positioned and oriented within the body cavity so as to be useable to cryoablate a tissue located at substantially any position within the cavity.

The substantially flexible medial portion may comprise a substantially rigid subsection, which may have a slip-resistant surface for facilitating grasping and maneuvering of the cryosurgery apparatus.

According to still further features in the described preferred embodiments, the medial portion comprises a medial heat exchanging configuration for exchanging heat between a first gas contained in the first medial conduit and a second gas contained in the second medial conduit. Preferably, the heat exchanging configuration is operable to transfer heat from a more highly compressed gas in the first conduit to a less highly compressed gas in the second conduit. Preferably, the heat exchanging configuration is also operable to transfer heat from a less highly compressed gas in the second conduit to a more highly compressed gas in the first conduit.

According to still further features in the described preferred embodiments, the cryosurgery apparatus further comprises a plurality of cryoprobes, and a plurality of surgical instruments.

According to still further features in the described preferred embodiments, the plurality of the surgical instruments comprises an ultrasound device, a thermal sensor, a camera, a grasping tool designed and constructed for grasping, holding, and moving the cryoprobe, and a tool selected from the group consisting of a grasping tool, an incising tool, a dissecting tool, a clipping tool, a cutting tool, a coagulating tool, and a sensor operable to report physically measurable data from a treated organ.

According to another aspect of the present invention there is provided a cryosurgery method for cryoablation of a tissue of a body, comprising installing at least a first trocar in a wall of a cavity of the body, introducing at least one substantially flexible cryoprobe into the cavity of the body through a portal of the first trocar, positioning and orienting the cryoprobe so that a cooling surface of the cryoprobe is adjacent to a tissue to be cryoablated, and cooling the cooling surface of the cryoprobe to cryoablation temperature and maintaining the cooling for a selected period of time, so as to cool the tissue to a cryoablation temperature, thereby cryoablating the tissue.

According to further features in preferred embodiments of the invention described below, the method further comprises heating the cooling surface to a selected temperature and maintaining the heating for a selected period of time, and further comprising multiple cycles of alternating cooling and heating of the cooling surface.

According to still further features in the described preferred embodiments, the method further comprises introducing a plurality of surgical tools through the portal of the installed first trocar, and introducing, through the portal of the installed first trocar, a surgical instrument selected from the group consisting of a grasping tool, an incising tool, a dissecting tool, a clipping tool, a cutting tool, a coagulating tool, and a sensor operable to report physically measurable data from a treated organ.

According to still further features in the described preferred embodiments, the method further comprises utilizing at least one of the plurality of surgical tools to position and orient the cryoprobe, utilizing at least one of the plurality of surgical tools to monitor positioning of the cryoprobe, utilizing at least one of the plurality of surgical tools to monitor temperature in a vicinity of the cryoprobe, and utilizing at least one of the plurality of surgical tools to monitor freezing of tissues in a vicinity of the cryoprobe.

According to still further features in the described preferred embodiments, the method further comprises introducing a plurality of substantially flexible cryoprobes into the cavity of the body through a portal of the trocar.

According to still further features in the described preferred embodiments, the method further comprises utilizing the plurality of cryoprobes to cryoablate a single tumor.

According to still further features in the described preferred embodiments, the method further comprises utilizing the plurality of cryoprobes to cryoablate a plurality of tumors.

According to still further features in the described preferred embodiments, the method further comprises installing a plurality of trocars in a wall of a cavity of the body, and introducing a plurality of substantially flexible cryoprobes into the cavity of the body, at least one of the plurality of cryoprobes being introduced through a portal of each of the plurality of trocars. The method further comprises utilizing the plurality of cryoprobes to cryoablate a single tumor, and utilizing the plurality of cryoprobes to cryoablate a plurality of tumors.

According to still further features in the described preferred embodiments, the method further comprises installing a second trocar in a wall of the body cavity, introducing at least one surgical tool into the body cavity through a portal of the second trocar, and utilizing the surgical tool to monitor positioning of the cryoprobe introduced into the body cavity through a portal of the first trocar.

According to still further features in the described preferred embodiments, the method further comprises introducing at least one surgical tool into the body cavity through a portal of the second trocar, and utilizing the surgical tool to monitor temperature in a vicinity of the cryoprobe introduced into the body cavity through a portal of the first trocar.

According to still further features in the described preferred embodiments, the method further comprises installing a second trocar in a wall of the body cavity, introducing at least one surgical tool into the body cavity through a portal of the second trocar, and utilizing the surgical tool to monitor freezing of tissues in a vicinity of the cryoprobe introduced into the body cavity through a portal of the first trocar.

According to yet another aspect of the present invention there is provided a flexible cryoprobe for cryoablating a tissue of a body, comprising a distal portion which comprises a tissue-cooling surface operable to cool a body tissue adjacent to the tissue-cooling surface and a cooling device for cooling the tissue-cooling surface, further comprising a substantially flexible medial portion designed and constructed for insertion into a body of a patient. The present invention successfully addresses shortcomings of the presently known configurations by providing an apparatus and method for cryoablation of tissues within a body cavity, enabling placement of cryoprobes in a variety of orientations and within substantially all regions of the cavity.

The present invention further successfully addresses shortcomings of the presently known configurations by providing an apparatus and method which enable use of multiple cryoprobes to affect cryoablation at one or more sites within a body cavity, and which enable use of cryoprobes in coordination with additional surgical tools useable to direct and to monitor placement of cryoprobes with respect to selected body tissues, and which further enable coordinated use of cryoprobes together with tools for monitoring progress of a cryoablation procedure as that procedure takes place.

The present invention further successfully addresses shortcomings of the presently known configurations by providing an apparatus and method enabling to perform cryoablation within a body cavity, such as the abdominal cavity, as part of a procedure which comprises inflating that body cavity with a gas under pressure, and maintaining that inflated state during the cryoablation phase of the procedure.

Implementation of the method and apparatus of the present invention involves performing or completing selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a simplified schematic of a cryoprobe utilizable to affect cryoablation, according to methods of prior art;

FIG. 2 is a simplified schematic of an arrangement for efficient connection of a plurality of cryoprobes to a source of compressed gas for use in a cryosurgical procedure, according to methods of prior art;

FIG. 3 is a simplified schematic of a pre-cooling element usable in an arrangement for connecting multiple cryoprobes to a common source of compressed gas, according to methods of prior art;

FIG. 4 is a simplified schematic of an apparatus utilizable for cryosurgery within a body cavity, according to an embodiment of the present invention;

FIG. 5 is a simplified schematic of a cryosurgery apparatus comprising a plurality of cryoprobes introduced into a body cavity through a common trocar, according to an embodiment of the present invention;

FIG. 6 is a simplified schematic of a configuration of surgical tools for use in a cryoablation procedure, according to an embodiment of the present invention;

FIG. 7 is a simplified schematic of a pressure-maintaining trocar with a removable diaphragm module, according to an embodiment of the present invention;

FIGS. 8A, 8B, and 8C are simplified schematic views of a diaphragm module of a pressure-maintaining trocar, according to an embodiment of the present invention;

FIG. 9 is a simplified cut-away view of a diaphragm module providing passage to a flexible cryoprobe, according to a preferred embodiment of the present invention;

FIG. 10 is a simplified schematic of a flexible maneuverable cryoprobe useable for performing cryosurgery through a trocar and within a body cavity, according to a preferred embodiment of the present invention; and

FIG. 11 is a simplified flow chart of a method for cryoablation within a body cavity, according to an embodiment of the present invention.

### DESCRIPTION OF THE PREFERRED EMBODIMENTS

The present invention relates to an apparatus and method for cryosurgery within a body cavity. More particularly, the present invention relates to an apparatus including a trocar having an edge shaped for penetrating an external wall of a body cavity, thereby opening an external passageway into that body cavity, and also having a portal serving to maintain and control that external passageway after installation of the trocar, the portal being useable for transmitting therethrough at least one surgical instrument for use during a surgical procedure. The device further includes at least one cryoprobe deployable through a portal of a trocar into a body cavity, which cryoprobe is operable to be positioned in that body cavity in a selected orientation and position, and is further operable to cryoablate a tissue within that body cavity when in a selected orientation and position.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice through which gas passes from the first, higher pressure, region of the device to the second, lower pressure, region of the device. It may further include a heat-exchanging configuration, for example a heat-exchanging configuration used to cool gasses from the first region of the device, prior to their expansion into the second region of the device. As is described hereinbelow, the expansion of certain gasses (referred to herein as “cooling gasses”) in a Joule-Thomson heat exchanger, when passing from a region of higher pressure to a region of lower pressure, causes these gasses to cool and may cause them to liquefy, creating a cryogenic pool of liquefied gas. This process cools the Joule-Thomson heat exchanger itself, and also cools any thermally conductive materials in contact therewith. As further described hereinbelow, the expansion of certain other gasses (referred to herein as “heating gasses”) in a Joule Thompson heat exchanger causes the gas to heat, thereby heating the Joule-Thomson heat exchanger itself and also heating any thermally conductive materials in contact therewith.

As used herein, a “Joule Thomson cooler” is a Joule Thomson heat exchanger used for cooling. As used herein, a “Joule Thomson heater” is a Joule Thomson heat exchanger used for heating.

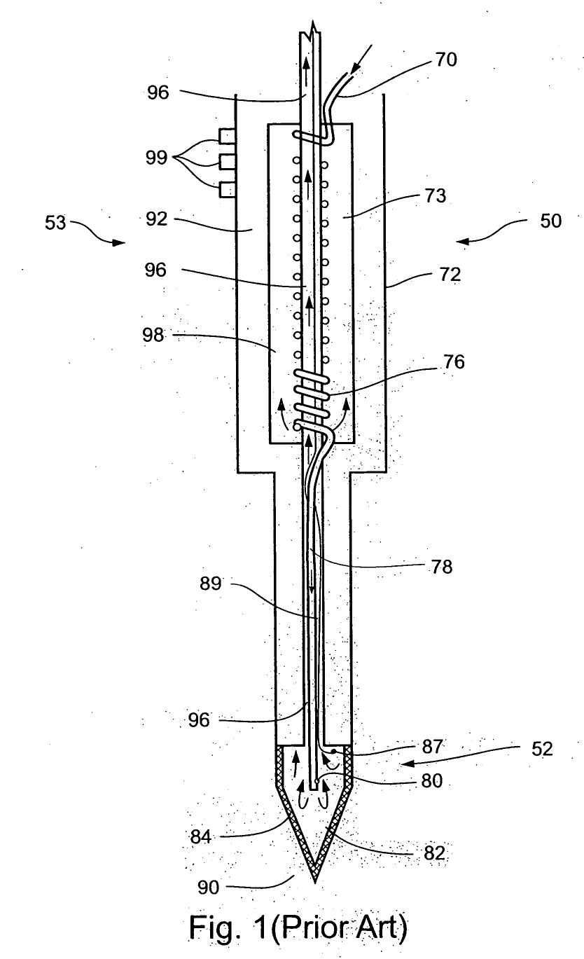

For purposes of better understanding the present invention, reference is first made to the construction and operation of a conventional (i.e., prior art) cryoprobe useable for affecting cryoablation of body tissue, as illustrated in FIGS. 1-3.

Attention is now drawn to FIG. 1, which presents a simplified schematic of a cryoprobe utilizable to affect cryoablation, according to a configuration of prior art.

As shown in FIG. 1, a cryoprobe 53 has an operating tip 52 including a Joule-Thomson cooler for freezing a patient's tissue, and a holding member 50 for holding by a surgeon. Operating tip 52 includes at least one passageway 78 extending therethrough for providing gas of high pressure to orifice 80 located at the end of operating tip 52, orifice 80 being for passage of high pressure gas therethrough, so as to cool operating tip 52 and produce an ice-ball at its end 90. Gases that may be used for cooling, referred to hereinafter as “cooling gasses”, include, but are not limited to, argon, nitrogen, air, krypton, CO₂, CF₄, xenon, and N₂O.

When a high pressure cooling gas such as argon expands through orifice 80, it cools and may liquefy so as to form a cryogenic pool within chamber 82 of operating tip 52. The cooled gas and/or cryogenic pool effectively cools surface 84 of operating tip 52. Surface 84 of operating tip 52 is preferably made of a heat conducting material such as metal so as to enable the formation of an ice-ball at end 90 thereof. Deep cooling of tissues within an ice-ball effects cryoablation of those tissues.

Alternatively, a high-pressure gas such as helium may be used for heating operating tip 52 via a reverse Joule-Thomson process. Gasses, such as helium, having this property are referred to hereinbelow as “heating gasses.” When a high-pressure heating gas expands through orifice 80 it heats chamber 82, thereby heating surface 84 of operating tip 52. Heating of a cryoprobe is useful in that it enables treatment by cycles of cooling-heating, and is further useful during extraction of a cryoprobe from tissue which has been frozen, since melting the contact interface between a probe and frozen body tissues prevents sticking of the probe to the tissue when the probe is extracted from the patient's body. Heating of a cryoprobe also enables fast extraction, when desired.

Operating tip 52 includes at least one evacuating passageway 96 extending therethrough for evacuating gas from operating tip 52 to the atmosphere.

As shown FIG. 1, holding member 72 may include a preliminary heat-exchanging configuration 73 for pre-cooling gas flowing through passageway 78. Specifically, the upper portion of passageway 78 may be in the form of a spiral tube 76 wrapped around evacuating passageway 96, the spiral tube being accommodated within a chamber 98. Thus, expanded cooling gas evacuated through passageway 96 may pre-cool incoming cooling gas flowing through spiral tube 76. Similarly, expanded heating gas evacuated through passageway 96 may pre-heat incoming heating gas flowing through spiral tube 76.

As further shown in FIG. 1, holding member 72 may include an insulating body 92 for thermally insulating heat-exchanging configuration 73 from the external environment.

Furthermore, operating tip 52 may include at least one thermal sensor 87 for sensing the temperature within chamber 82, the wire 89 of which extending through evacuating passageway 96 or a dedicated passageway (not shown).

In addition, holding member 72 may include a plurality of switches 99 for manually controlling the operation of probe 53 by a surgeon. Such switches may provide functions such as on/off, heating, cooling, and predetermined cycles of heating and cooling by selectively and controllably communicating incoming passageway 70 with an appropriate external gas container including a cooling or a heating gas.

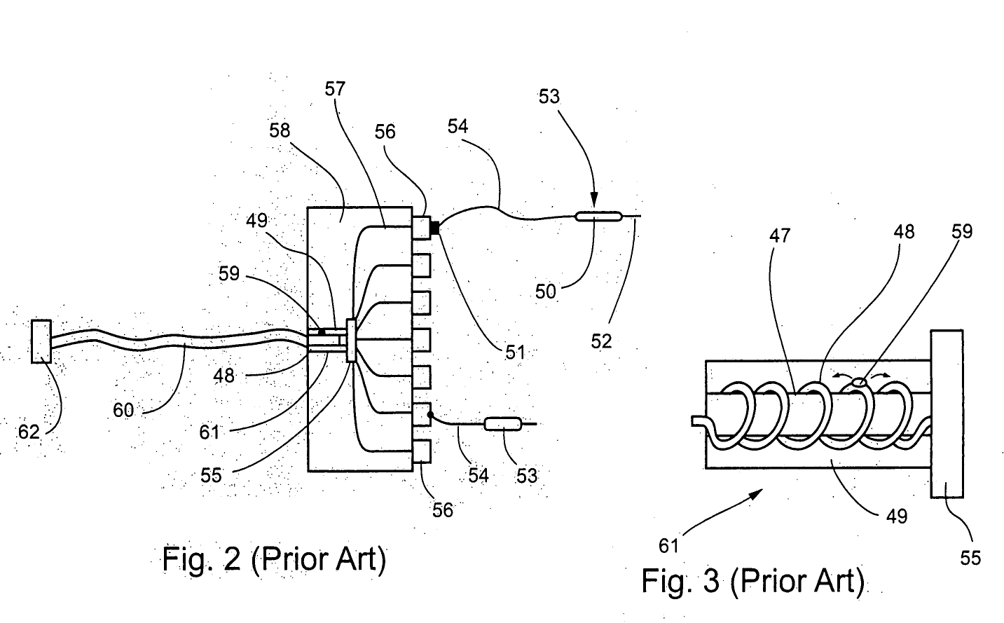

Attention is now drawn to FIG. 2, which is a simplified schematic of an arrangement for efficient connection of a plurality of cryoprobes for use in a cryosurgical procedure, according to a configuration of prior art. FIG. 2 presents a plurality of cryosurgical probes 53 connected via a flexible connecting line 54 to a connecting site 56 on a housing element 58, preferably by means of a linking element 51. Cryosurgical probes 53 may be detachably connected to connecting sites 56.

Preferably, evacuating passageway 96 extends through connecting line 54, such that the outgoing gas is evacuated through an opening located at linking element 51 or at any other suitable location, e.g., manifold 55, see below. Preferably, line 54 further includes electrical wires for providing electrical signals to the thermal sensor and switches (not shown).

Each of cryosurgical probes 53 is in fluid communication with a manifold 55 received within a housing 58, manifold 55 being for distributing the incoming high pressure gas via lines 57 to cryosurgical probes 53.

As shown, housing 58 is connected to a connector 62 via a flexible cable 60 including a gas tube (not shown), connector 62 being for connecting the apparatus to a high-pressure gas source and an electrical source.

The apparatus further includes electrical wires (not shown) extending through cable 60 and housing 58 for providing electrical communication between the electrical source and cryosurgical probes 53.

Preferably, housing 58 includes a pre-cooling element, generally designated as 61, for pre-cooling the high-pressure gas flowing to cryosurgical probes 53. Preferably, pre-cooling element 61 is a Joule-Thomson cooler, including a tubular member 48 received within a chamber 49, tubular member 48 including an orifice 59 for passage of high pressure gas therethrough, so as to cool chamber 49, thereby cooling the gas flowing through tubular member 48 into manifold 55.

Attention is now drawn to FIG. 3, which is a simplified schematic of an alternate configuration of a pre-cooling element used in connecting multiple cryoprobes to a common source of compressed gas, according to a configuration of prior art. As shown in FIG. 3, tubular member 48 is in the form of a spiral tube wrapped around a cylindrical element 47, so as to increase the area of contact between tubular member 48 and the cooling gas in chamber 49.

According to yet another configuration (not shown), housing 58 includes a first tubular member for supplying a first high pressure gas to manifold 55, and a second tubular member for supplying a second high pressure gas to pre-cooling element 61. Any combination of gases may be used for cooling and/or heating the gases flowing through such tubular members.

Alternatively, a cryogenic fluid such as liquid nitrogen may be used for pre-cooling the gas flowing through housing 58. Alternatively, an electrical pre-cooling element may be used for pre-cooling the gas.

Preferably, thermal sensors (not shown) may be located within cable 60 and manifold 55 for measuring the temperature of gas flowing therethrough.

The principles and operation of an apparatus for performing cryosurgery within a body cavity according to the present invention will now be explained with particular reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

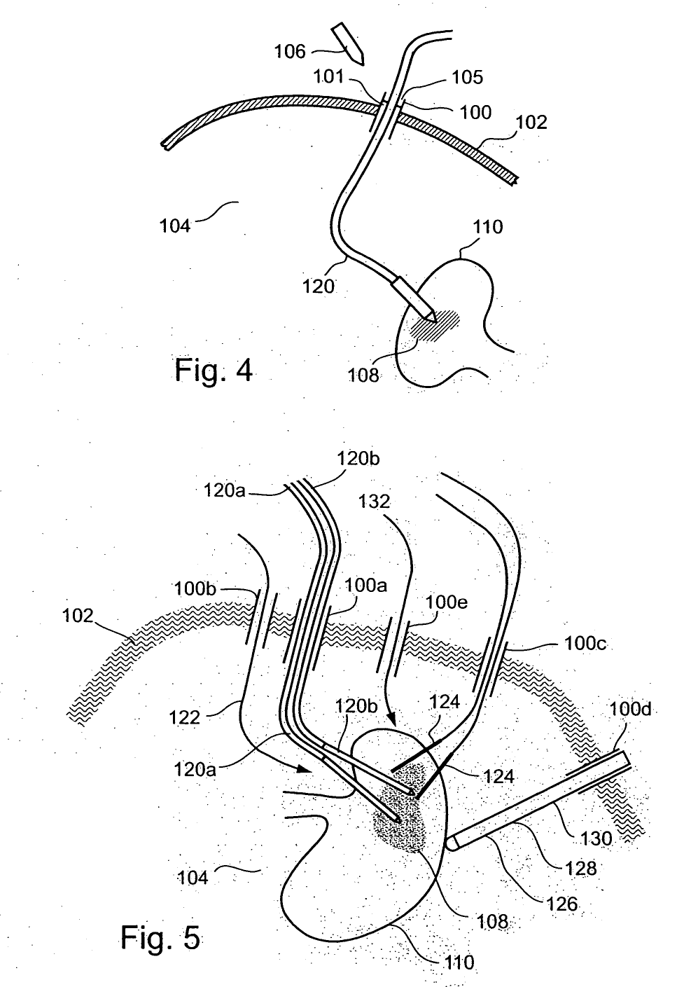

Reference is now made to FIG. 4, which is a simplified schematic of an apparatus utilizable for cryosurgery within a body cavity, according to an embodiment of the present invention.

FIG. 4 presents a trocar 100 positioned in an external passageway 101 opened in a wall 102 of a body cavity 104. Optionally detachable cutting element 106, used to effect penetration of trocar 100 through wall 102 is shown removed from trocar 100. A flexible maneuverable cryoprobe 120 is shown passing through a portal 105 of trocar 100 into body cavity 104. During use, cryoprobe 120 is appropriately positioned within body cavity 104 so as to be able to effect cryoablation of pathological tissue, such as a tumor, at a desired cryoablation site 108 within a body organ 110.

Reference is now made to FIG. 5, which is a simplified schematic of a cryosurgery apparatus comprising a plurality of cryoprobes introduced into a body cavity through a common trocar, according to a preferred embodiment of the present invention.

In FIG. 5, 120a and 120b are flexible maneuverable cryoprobes passing through a common trocar 100a into body cavity 104. In a currently preferred and recommended procedure, a grasping tool 122 is introduced into body cavity 104 through an additional trocar here designated 100b, or through a similar device. Grasping tool 122 is useable to grasp cryoprobes 120, to move them within body cavity 104, to exert pressure to effect penetration of tissues within body cavity 104 (for example, to affect penetration of organ 110), and in general to position cryoprobes 120 in a manner which is appropriate for cryoablation at desired cryoablation site 108.

As further shown in FIG. 5, in a currently preferred and recommended procedure, additional surgical instruments are introduced into body cavity 104 for monitoring positioning of cryoprobes 120, and for monitoring progress of cryoablation during a cryoablation procedure. For example, thermal sensors 124 are introduced in the vicinity of cryoablation site 108, enabling monitoring of temperature in proximity to cryoprobes 120, and an imaging device 126, such as an ultrasound probe 128 or an optical device 130, may be utilized to monitor the process of tissue freezing during a cryoablation procedure. Various other surgical instruments 132 may be utilized as well.

In FIG. 5, a plurality of cryoprobes 120 are shown as passing through a common trocar 10a, grasping tool 122 is shown passing into body cavity 104 through a second trocar 100b, thermal sensors 124 are shown passing through a third trocar 100c, imaging device 126 is shown passing through an additional trocar 100d, and additional unspecified surgical tools are shown passing through yet another trocar 10e. It is noted that the particular selection and arrangement of surgical tools depicted in FIG. 5 is provided by way of example, and is a currently preferred utilization of the embodiment here presented, yet is not limiting. A greater or lesser number of additional surgical tools may be utilized together with cryoprobes 120. Such tools may be introduced into body cavity 104 through a common trocar together with one or more cryoprobes 120, or may alternatively be introduced into body cavity 104 through additional trocars, or through ports of other sorts, through open incisions, by direct penetration of external wall 102. Indeed, some tools used in conjunction with cryoprobes 120, (for example, imaging devices such as roentgen or ultrasound) may yet be positioned external to body cavity 104.

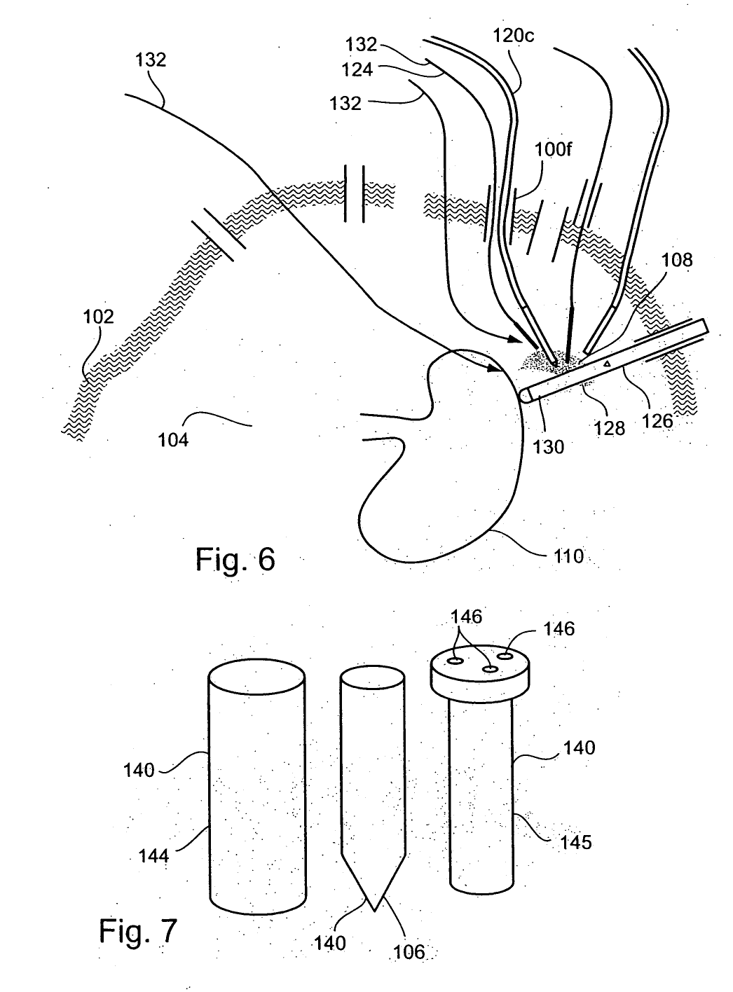

Attention is now drawn to FIG. 6, which is a generalized schematic of a configuration of surgical tools for use in a cryoablation procedure, according to an additional preferred embodiment of the present invention.

FIG. 6 provides additional examples of tool configurations. In particular, FIG. 6 presents a cryoprobe 120c and an additional surgical tool 132, such as a thermal sensor 124, introduced into body cavity 104 through a common trocar 100f. A variety of other surgical instruments, for use in conjunction with cryoprobe 120, are shown penetrating into body cavity 104 through additional trocars and through additional ports and openings of various sorts. Additional surgical tools 132 may include tools for all variety of surgical activities, including grasping, incision, dissection, clipping, cutting, coagulation, and so on, as well as sensors of various sorts for accumulating and reporting all kinds of physical measurable data from the treated organ and its surroundings, providing, for example, real-time feedback to a surgeon. Such sensors may also be used to provide real-time feedback to a control algorithm running on a computer control system useable to manage operation of a cryoprobe, e.g., by controlling supply of high-pressure gas to a cryoprobe as a function of measured and reported temperature at a site within the body of a patient.

Surgical tools 132 may also include tools for enabling enhanced visualization of a surgical procedure, such as a CCD or other type of television camera for creating still images or moving pictures of the interior of body cavity 104 during a surgical procedure. Such tools may be used, for example, to enhance critiques of on-going procedures, and to enhance teaching of the methodology. They the images thus produced may be recorded for subsequent playback and study, or ongoing audio and video input from within body cavity 104 can be delivered to remote viewers and/or proctors. In a teaching context, this information may further be edited, for example, by the addition of additional lines of drawing added to a displayed video image.

Attention is now drawn to FIG. 7, which is a simplified schematic of a pressure-maintaining trocar having a removable diaphragm module, according to a preferred embodiment of the present invention.

Establishing and maintaining gas pressurization within a body cavity is a frequently used procedure in endoscopic surgery. Typically, a neutral gas such as CO₂ is introduced through a trocar into a body cavity such as the abdominal cavity, where it serves to inflate the cavity, thereby separating the cavity walls from the internal organs contained in the cavity. Surgical manipulations within the cavity are easier to accomplish when the cavity is thus inflated, and visibility within the cavity (through utilization of cameras or optical visualization tools) is also much facilitated by the inflated state.

FIG. 7 presents a pressure-maintaining trocar 140 useable for introducing cryoablation tools into a body cavity and utilizing those tools within that body cavity, while maintaining a pressurization state within the cavity. As shown in FIG. 7, pressure-maintaining trocar 140 has a removable cutting element 106 and a removable diaphragm module 145, alternatively installable within an external tube body 144.

In typical use, prior to installation of trocar 140, cutting element 106 is mounted within external tube body 144 of trocar 140, thereby providing trocar 140 with a cutting edge suitable to effect an opening into a body cavity 104 through an external wall 102 of cavity 104. Cutting element 106, mounted in trocar 140, is pushed through wall 102, thereby creating an external passageway 101 into cavity 104 (not shown) and installing external tube body 144 of trocar 140 in an external passageway 101 thus created.

According to a currently preferred method of use, trocar 140 having been thus installed, removable cutting element 106 may then be removed from external tube body 144, and removable diaphragm module 145 may then be inserted into external tube body 144 in place of cutting element 106.

Diaphragm module 145, inserted in an external tube body 144 which is installed in external passageway 101, constitutes a portal operable to maintain and control external passageway 101. Moreover, in a preferred embodiment presented in FIG. 7, the fit of diaphragm module 140 within external tube body 144 is such as to provide a pressure-maintaining (e.g, airtight) seal. Diaphragm module 145 is provided with one or more diaphragms 146. Each diaphragm 146 is designed and constructed, preferably of rubber or a rubberized material, to permit passage therethrough of a cryoprobe or other surgical instrument, while yet maintaining a pressure-maintaining seal around that cryoprobe or other instrument. An unused diaphragm 146, that is, a diaphragm 146 through which no instrument passes, is similarly airtight. Thus, pressure maintaining trocar 140 is designed and constructed is such a way that, when pressure maintaining trocar 140 is installed in a wall of a body cavity, if a gas under pressure is introduced into that body cavity (either through a diaphragm 146 of diaphragm module 145 of trocar 140, or through some other opening), then trocar 140 is substantially able to maintain gas pressurization within body cavity 104, while yet allowing passage of one or more cryoprobes and/or other surgical instruments into that pressurized body cavity, thereby enabling execution of cryoablation procedures under favorable conditions of maneuverability of tools and visibility of objects within cavity 104.

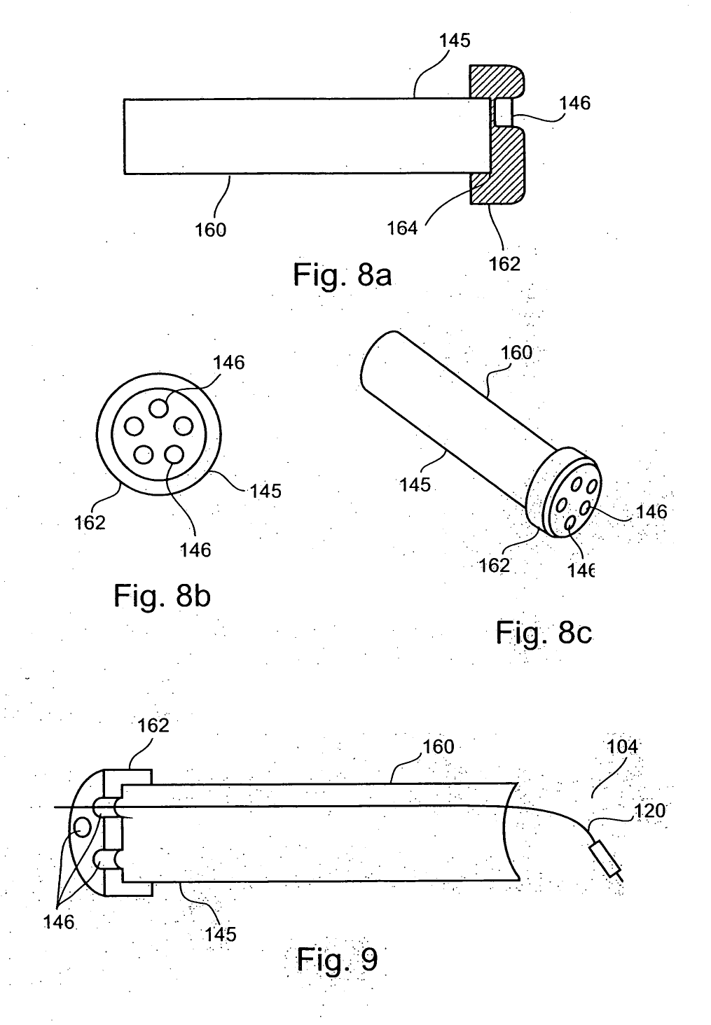

Attention is now draw to FIGS. 8A, 8B, and 8C, which are simplified schematic views of diaphragm module 145 of trocar 140, according to an embodiment of the present invention.

FIG. 8A provides a side view of diaphragm module 145, comprising a tubular section 160 and a bushing 162. Tubular section 160 is preferably glued to bushing 162 at surface 164. Bushing 162 comprises one or more diaphragms 146, and preferably a plurality of diaphragms 146, for passing surgical tools through bushing 162. A cryoprobe or other surgical tool passing through diaphragm 146 can be extended thence through tubular section 160 and on into a body cavity 104 in a wall of which trocar 140, containing diaphragm module 145, has been installed. If body cavity 104 is pressurized, each diaphragm 146 maintains a substantially airtight seal around a cryoprobe or other surgical tool passing therethrough. Similarly, diaphragm 146 also maintains a substantially airtight seal when no tool passes therethrough.

FIG. 8B provides a front view of diaphragm module 145. In this view, bushing 162 is shown having a plurality of diaphragms 146. FIG. 8C provides a perspective view of diaphragm module 145, also showing a plurality of diaphragms 146 in bushing 162.

Attention is now drawn to FIG. 9, which is a simplified cut-away view of a diaphragm module providing passage to a flexible maneuverable cryoprobe, according to a preferred embodiment of the present invention. In FIG. 9, a flexible maneuverable cryoprobe 120 is shown passing through a diaphragm 146 in bushing 162 of a diaphragm module 145, extending through tubular section 160 of diaphragm module 145, and extending on into a body cavity 104, where it may be used for cryoablation. As described hereinabove, additional diaphragms 146 in bushing 162 may be used to provide passage to additional cryoprobes 120, or to surgical tools of other sorts.

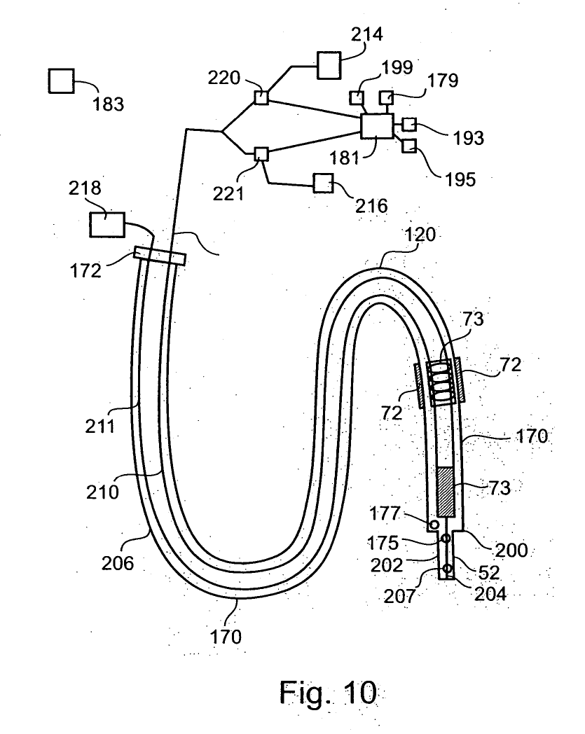

Attention is now drawn to FIG. 10, which is a simplified schematic of a flexible maneuverable cryoprobe useable for performing cryosurgery through a trocar and within a body cavity, according to a preferred embodiment of the present invention.

Flexible maneuverable cryoprobe 120 has a distal portion 200 which comprises a tissue-cooling surface 202 operable to cool adjacent body tissues, and a cooling device 204 for cooling that tissue-cooling surface. Flexible maneuverable cryoprobe 120 also has a substantially flexible medial portion 206 designed and constructed for insertion into a body cavity.

Flexible maneuverable cryoprobe 120 is preferably constructed according to the general principles elucidated hereinabove, particularly with reference to FIG. 1. In particular, in a preferred embodiment, distal portion 200 of flexible maneuverable cryoprobe 120 has an operating tip 52 including a Joule-Thomson heat exchanger 207 for freezing and optionally heating a patients tissue, including a gas delivery and evacuation system as described hereinabove with reference to FIG. 1.

It is noted, however, that alternate constructions may also be utilized. In one alternate construction, a plurality of Joule-Thomson coolers may be used.

In another alternate construction, flexible maneuverable cryoprobe 120 may be designed and constructed to be cooled by methods other than Joule-Thomson cooling, for example by evaporation of liquefied gasses such as N₂ or CO₂.

Similarly, flexible maneuverable cryoprobe 120 may alternatively be designed and constructed to be heated by methods other than Joule-Thomson heating, such as, for example, by electrical heating, or by externally electrically heated low pressure gas.

In a preferred embodiment having a Joule-Thomson heat exchanger in operating tip 52, flexible medial portion 206 comprises a first medial conduit 210 for transmitting a gas from a source of compressed gas to distal portion 200. In a particularly preferred embodiment, first medial conduit 210 is operable to transmit gas from a plurality of sources of compressed gas to said distal portion. As shown in FIG. 10, a pressurized cooling-gas source 214 may be connected to probe 120 through cooling gas valve 220 and a gas connector 172, and a pressurized heating gas source 216 may be connected to probe 120 through heating gas valve 221 and gas connector 172, thereby providing sources of both cooling gas and heating gas to Joule-Thomson heat exchanger 207 in distal portion 200. In this preferred embodiment, medial portion 206 further comprises a second medial conduit 211 for transmitting gas after depressurization in Joule-Thomson heat exchanger 207, to a gas receiving unit 218 positioned outside said body cavity.

Flexible maneuverable cryoprobe 120 is distinguished from cryoprobes known to prior art by the presence of flexibility module 170, flexibly linking operating tip 52 to a source of compressed gas through gas connector 172. Flexibility module 170 is designed and constructed for penetration into body cavity 104 through trocar 100, and preferably through a diaphragm 146 of diaphragm module 145 of pressure-maintaining trocar 140. Flexibility module 170 enables substantially free maneuverability of cryoprobe 120 within body cavity 104. Thus, flexibility module 170 of flexible maneuverable cryoprobe 120 enables a surgeon, having introduced cryoprobe 120 into a body cavity 104 of a patient, to then maneuver cryoprobe 120 to substantially any desired position and orientation within cavity 104. Flexibility module 170 may optionally include one or more rigid sections alternating with one or more flexible sections. A rigid heat-exchanging configuration 73, for example, may be placed at one or more points within an otherwise substantially flexible flexibility module 170.

Cryoprobe 120 optionally includes one or more holding members 72 for convenient grasping and manipulation of cryoprobe 120 by a surgeon using grasping tools of various sorts. A holding member 72 may comprise, for example, hard or resistant surfaces, or special perforations, groves, holes or similar structures, to allow for easy and slip-resistant grasping and maneuvering of various portions of cryoprobe 120 while cryoprobe 120 is within body cavity 104.

Cryoprobe 120 also optionally includes one or more preliminary heat-exchanging configuration 73, whose function, as described hereinabove with reference to FIG. 1, is to provide for pre-cooling of cooling gasses being delivered to operating tip 52, or alternatively to provide for pre-heating of heating gasses being delivered to operating tip 52. Preliminary heat-exchanging configuration 73 may be constructed in close proximity to operating tip 52, in a manner similar to that shown in FIG. 1. It is to be noted, however, that preliminary heat-exchanging configuration 73 may alternatively be constructed distant from operating tip 52, and be connected to operating tip 52 by means of an additional flexibility module 170 linking preliminary heat-exchanging configuration 73 to operating tip 52.

Distal portion 200 of cryoprobe 120 is preferably of metal construction. Distal portion 200 is preferably short, between 1 and 10 cm long, and most preferably is of 5 cm length, or less. The width of cryoprobe 120 is preferably between 1 and 5 mm, and preferably between 1 and 1.5 mm. (Width of probe 120 has been exaggerated in FIG. 10, for clarity.)

In an alternate configuration, flexible maneuverable cryoprobe 120 may be designed and constructed having a distal portion comprising an introducer sufficiently sharp so as to able to penetrate into a tissue such as a body organ, the introducer being designed and constructed to contain a plurality of operating tips 52, those operating times being deployable from that introducer into tissues surrounding that introducer when the introducer is positioned in proximity to a tissue to be cryoablated.

In a further alternate configuration, flexible maneuverable cryoprobe 120 may include a data sensor 175 operable to supply real-time status information, such as temperature information, through a wire (not shown) or through a wireless communication module 177, to an external receiver 179 operable to pass such information to a control computer 181 or a display 183.

In a preferred embodiment, control computer 181 is operable to open and close cooling gas valve 220 and heating gas valve 221. Valves 220 and 221, under control of control computer 181, or alternatively manually or electrically controlled by a human operator, are useable to selectively control flow of cooling and heating gas to Joule-Thomson heat exchanger 207 in operating tip 52, and thereby to control heating and cooling of operating tip 52.

Computer 181 preferably comprises a memory module 193, such as a hard disk, useable to record operational data. Computer 181 is preferably controlled by a programmed operating algorithm 195, which generates operational commands to valves 220 and 221, under general guidance of a human operator who interfaces with computer 181 through input/output interface 199.

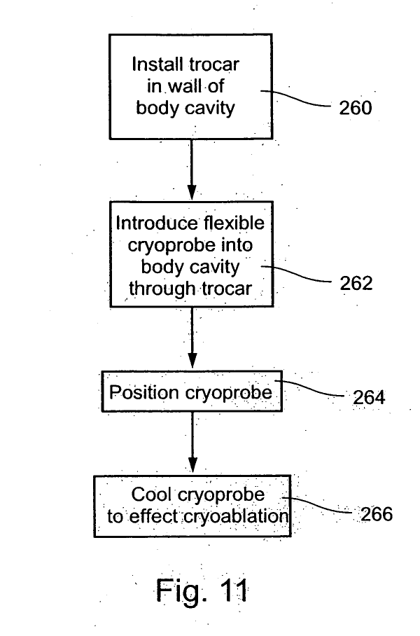

Attention is now drawn to FIG. 11, which is a simplified flow chart of a method for cryoablation within a body cavity, according to an embodiment of the present invention.

As shown in FIG. 11, at step 260, a trocar is installed in a wall of a body cavity. Alternatively, a plurality of trocars may be so installed.

At step 262, a substantially flexible cryoprobe, such as the cryoprobe described hereinabove with reference to FIG. 10, is introduced into the body cavity through a portal of the installed trocar. Alternatively, a plurality of cryoprobes may be introduced, either through a same trocar or through a plurality of installed trocars. Additional surgical tools may also be introduced into the body cavity in similar manner, either through a trocar through which a cryoprobe is introduced, or through a separate trocar. Tools usefully introduced in this manner include a grasping tools, incising tools, dissecting tools, clipping tools, cutting tools, coagulating tools, and sensors, such as sensors operable to report physically measurable data from a treated organ, for example temperature sensors.

At step 264, a cryoprobe introduced into a body cavity in step 262 is positioned and oriented within the body cavity so that a cooling surface of that introduced cryoprobe is adjacent to a tissue to be cryoablated. In a recommended procedure, a grasping tool introduced into the body cavity at step 262 may be used at step 264 to manipulate introduced cryoprobes, navigating them to a desired position and orientation. Similarly, observation tools, such as a camera or optical viewer, may be introduced at step 262, and utilized at step 264 to monitor and report on positioning of cryoprobes being manipulated into a desired position.

At step 266, a cooling surface of an introduced and positioned cryoprobe is cooled to cryoablation temperature, which temperature is maintained for a selected period of time. Tools introduced at step 262 may be used to monitor temperature in a vicinity of a cooled cryoprobe, or to monitor freezing of tissues during cooling. Ultrasound sensing, for example, may be used to monitor freezing of tissues.

As a result of steps 260, 262, 264, and 266, tissue adjacent to an introduced and positioned cryoprobe is cooled to cryoablation temperature, thereby cryoablating that tissue.

Additionally, a cooling surface may be heated after being used to cryoablate a tissue, for example to facilitate extraction of a cryoprobe from a cryoablation site. Similarly, a cryoprobe may be alternatingly cooled and heated in repeated cycles. Multiple cryoprobes introduced according to the method outlined in FIG. 11 may be used to cryoablate a single tumor, or to cryoablate a plurality of tumors. According to the convenience of an operating surgeon, multiple cryoprobes introduced through a single trocar may be utilized to cryoablate either a single tumor or multiple tumors, and multiple cryoprobes introduced through multiple trocars may similarly be utilized to cryoablate either a single tumor or multiple tumors.

Similarly, when a cryoprobe is introduced into a body cavity through a first trocar, a surgical tool introduced either through a same trocar or through an additional trocar may be used to monitor positioning of that introduced cryoprobe, or to monitor temperatures in a vicinity of that introduced trocar, or to monitor freezing of tissues in a vicinity of that introduced trocar.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A cryosurgery method for cryoablation of a tissue of a body, comprising:
(a) installing at least a first trocar in a wall of a cavity of said body;
(b) introducing at least one substantially flexible cryoprobe into said cavity of said body through a portal of said first trocar;
(c) positioning and orienting said cryoprobe so that a cooling surface of said cryoprobe is adjacent to a tissue to be cryoablated; and
(d) cooling said cooling surface of said cryoprobe to cryoablation temperature and maintaining said cooling for a selected period of time;
thereby cooling said tissue to a cryoablation temperature, thereby cryoablating said tissue.

2. The method of claim 1, further comprising heating said cooling surface to a selected temperature and maintaining said heating for a selected period of time.

3. The method of claim 2, further comprising multiple cycles of alternating cooling and heating of said cooling surface.

4. The method of claim 1, further comprising introducing a plurality of surgical tools through said portal of said installed first trocar.

5. The method of claim 1, further comprising introducing, through said portal of said installed first trocar, a surgical instrument selected from the group consisting of a grasping tool, an incising tool, a dissecting tool, a clipping tool, a cutting tool, a coagulating tool, and a sensor operable to report physically measurable data from a treated organ.

6. The method of claim 4, further comprising utilizing at least one of said plurality of surgical tools to position and orient said cryoprobe.

7. The method of claim 4, further comprising utilizing at least one of said plurality of surgical tools to monitor positioning of said cryoprobe.

8. The method of claim 4, further comprising utilizing at least one of said plurality of surgical tools to monitor temperature in a vicinity of said cryoprobe.

9. The method of claim 4, further comprising utilizing at least one of said plurality of surgical tools to monitor freezing of tissues in a vicinity of said cryoprobe.

10. The method of claim 1, further comprising introducing a plurality of substantially flexible cryoprobes into said cavity of said body through a portal of said trocar.

11. The method of claim 10, further comprising utilizing said plurality of cryoprobes to cryoablate a single tumor.

12. The method of claim 10, further comprising utilizing said plurality of cryoprobes to cryoablate a plurality of tumors.

13. The method of claim 1, further comprising installing a plurality of trocars in a wall of a cavity of said body, and introducing a plurality of substantially flexible cryoprobes into said cavity of said body, at least one of said plurality of cryoprobes being introduced through a portal of each of said plurality of trocars.

14. The method of claim 13, further comprising utilizing said plurality of cryoprobes to cryoablate a single tumor.

15. The method of claim 10, further comprising utilizing said plurality of cryoprobes to cryoablate a plurality of tumors.

16. The method of claim 1, further comprising installing a second trocar in a wall of said body cavity, introducing at least one surgical tool into said body cavity through a portal of said second trocar, and utilizing said surgical tool to monitor positioning of said cryoprobe introduced into said body cavity through a portal of said first trocar.

17. The method of claim 1, further comprising installing a second trocar in a wall of said body cavity, introducing at least one surgical tool into said body cavity through a portal of said second trocar, and utilizing said surgical tool to monitor temperature in a vicinity of said cryoprobe introduced into said body cavity through a portal of said first trocar.

18. The method of claim 1, further comprising installing a second trocar in a wall of said body cavity, introducing at least one surgical tool into said body cavity through a portal of said second trocar, and utilizing said surgical tool to monitor freezing of tissues in a vicinity of said cryoprobe introduced into said body cavity through a portal of said first trocar.

19. A flexible cryoprobe for cryoablating a tissue of a body, comprising:
(a) a distal portion which comprises
(i) a tissue-cooling surface operable to cool a body tissue adjacent to said tissue-cooling surface; and
(ii) a cooling device for cooling said tissue-cooling surface; and
(b) a substantially flexible medial portion designed and constructed for insertion into a body of a patient.
